(19) United States
(12) Patent Application Publication
(10) Pub. No.: US 2017/0360277 A1
(43) Pub. Date: Dec. 21, 2017

(54) MEDICAL POWER SUPPLY SYSTEM

(71) Applicant: OLYMPUS CORPORATION, Tokyo (JP)

(72) Inventors: Kaoru MATSUKI, Kawasaki-shi (JP); Akira MATSUI, Tokyo (JP); Shoei TSURUTA, Tokyo (JP)

(73) Assignee: OLYMPUS CORPORATION, Tokyo (JP)

(21) Appl. No.: 15/679,715

(22) Filed: Aug. 17, 2017

Related U.S. Application Data
(63) Continuation of application No. PCT/JP2015/055876, filed on Feb. 27, 2015.

(51) Int. Cl. A61B 1/00; A61B 18/14; A61B 17/02; A61B 17/34; H01F 38/14; H02J 50/10; A61B 18/12; A61B 17/00; A61B 17/29

(52) U.S. Cl. CPC A61B 1/00029; H01F 38/14; A61B 18/1442; A61B 1/00124; A61B 1/00135; A61B 17/0218; A61B 18/1482; H02J 50/10; A61B 17/3476; A61B 17/3498; A61B 2017/00398; A61B 2017/2925; A61B 2018/1286

(57) ABSTRACT

A medical power supply includes a medical instrument and a guide tube. The power receiving member is arranged radially outside the insertion portion, and when the insertion portion is inserted into the guide tube to a predetermined length such that the power transmitting member and the power receiving member are close to each other, a power transmitting side proximal end surface of the power transmitting member and a power receiving side distal end surface of the power receiving member face each other in an axial direction of the insertion portion and are in a positional relationship such that wireless power supply is possible.

This application is a continuation based on PCT/JP2015/055876, filed on Feb. 27, 2015. The content of the United States Patent Application is incorporated herein by reference.

### TECHNICAL FIELD

The present invention relates to a medical power supply system.

### BACKGROUND ART

In the related art, it is known that various treatments are performed on a patient while minimizing stress using a trocar and a medical instrument.

The inner needle punctures a body wall of a patient and a trocar is inserted into an abdominal cavity in the state where the trocar is integrally combined with the inner needle having a sharp puncturing portion at the tip. After the trocar is inserted into the abdominal cavity, the inner needle is removed such that the trocar is placed in the body wall and the trocar is then used as a guide tube for a treatment tool that performs a treatment in the abdominal cavity.

As a medical instrument, the treatment tool is inserted into the body of a patient via the trocar. In the case where the treatment tool uses electricity to perform treatments, a cable for supplying power is usually connected to the treatment tool. This cable reduces the manipulability of the medical instrument when an operator performs treatments.

In this regard, a technology in which wireless power supply is performed between a trocar and a medical instrument to eliminate the necessity of a cable is disclosed in Japanese Patent Granted Publication No. 4145395.

In the technology described in Patent Literature 1, a primary coil (a power transmitting coil) provided at a trocar and a secondary coil (a power receiving coil) provided at a treatment tool which has been inserted into the trocar are electromagnetically coupled, thereby, it is possible to perform wireless power supply from the trocar to the treatment tool.

### SUMMARY OF INVENTION

A medical power supply system according to a first aspect of the present invention includes a medical instrument which has an elongated insertion portion that is inserted into a body, an end effector that is provided at a distal end portion of the insertion portion, and a power receiver that has a power receiving member and receives power to be supplied to the end effector; and a guide tube which has a power transmitting member that is connected to a power source and that is provided at a proximal end side of the guide tube, and which guides the insertion portion of the medical instrument so as to be inserted from the proximal end side into the body. The power receiving member is arranged radially outside the insertion portion, and when the insertion portion is inserted into the guide tube to a predetermined length such that the power transmitting member and the power receiving member are close to each other, a power transmitting side proximal end surface of the power transmitting member and a power receiving side distal end surface of the power receiving member face each other in an axial direction of the insertion portion and are in a positional relationship such that wireless power supply is possible.

### BRIEF DESCRIPTION OF DRAWINGS

FIG. 1 is a side view illustrating a treatment tool that is a medical instrument of a medical power supply system according to a first embodiment of the present invention.

FIG. 2 is a front view of the treatment tool shown in FIG. 1.

FIG. 3 is a side view illustrating a trocar that is a guide tube of the medical power supply system according to the first embodiment of the present invention.

FIG. 4 is a plan view of the trocar shown in FIG. 3.

FIG. 5 is a view illustrating a procedure when the medical power supply system according to the first embodiment of the present invention is used.

FIG. 6 is a view illustrating a procedure when the medical power supply system according to the first embodiment of the present invention is used.

FIG. 7 is a schematic diagram illustrating the relationship between the sizes of a power receiving member and a power transmitting member of the medical power supply system according to the first embodiment of the present invention.

FIG. 8 is a perspective view illustrating a modified example of the medical power supply system according to the first embodiment of the present invention.

FIG. 9 is a side view illustrating the modified example of the medical power supply system according to the first embodiment of the present invention.

FIG. 10 is a side view illustrating a treatment tool that is a medical instrument of a medical power supply system according to a second embodiment of the present invention.

### DESCRIPTION OF EMBODIMENTS

### First Embodiment

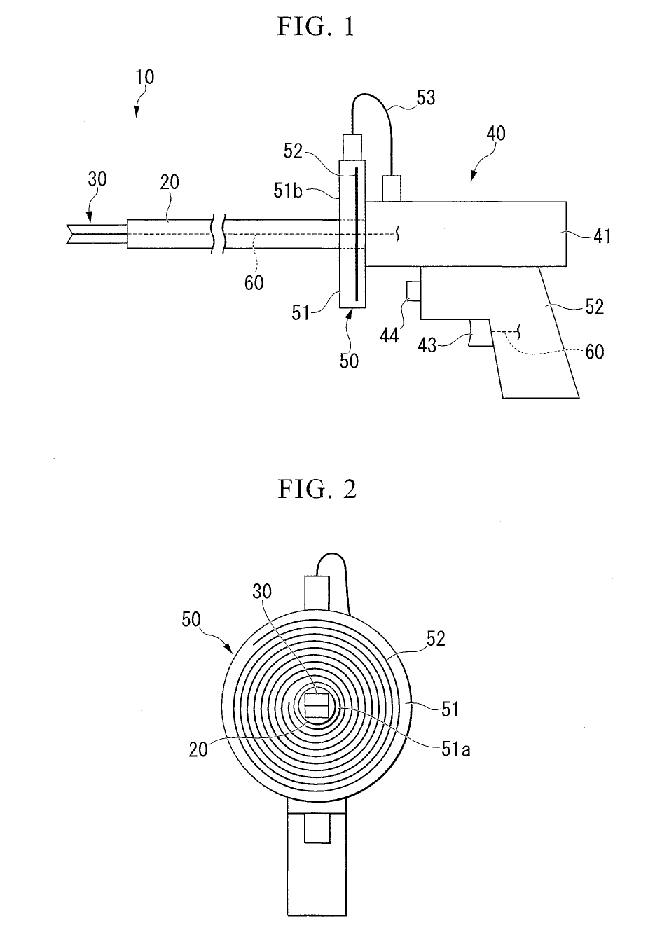

Hereinafter, a first embodiment of the present invention is described with reference to FIGS. 1 to 4. A medical power supply system of the present embodiment includes a trocar (a guide tube) that is placed into a patient or the like and a medical instrument that is inserted and used in the trocar.

FIG. 1 is a side view illustrating a treatment tool 10 that is a medical instrument of the present embodiment. FIG. 2 is a front view of the treatment tool 10.

The treatment tool 10 includes an insertion portion 20, a treatment portion (an end effector) 30, a manipulation portion 40, and a power receiver 50. The insertion portion 20 is formed of metal, resin, or the like in the shape of an elongated tube and at least a part of the insertion portion 20 is inserted together with the treatment portion 30 into a body.

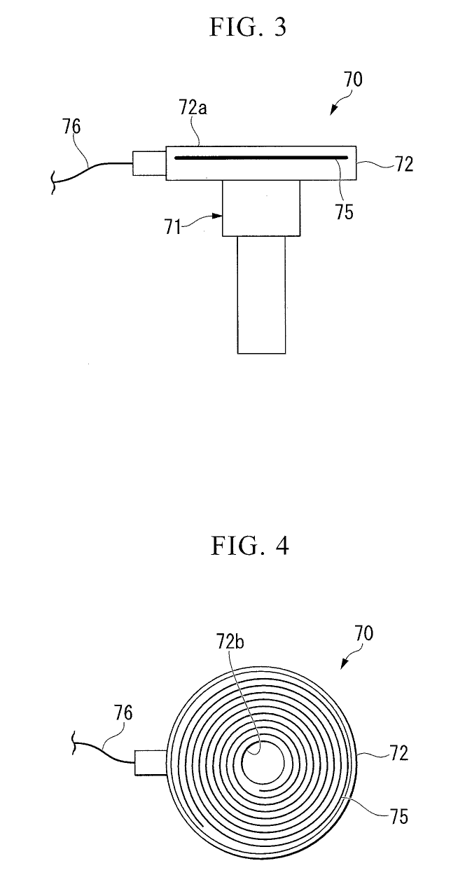

In the description of the present specification, one end of the treatment tool 10 at which the manipulation portion 40 is provided and one end of a trocar 70 shown in FIG. 3 from which the insertion portion 20 of the treatment tool 10 is inserted into a body are each referred to as a “proximal end”.

The treatment portion 30 is provided at a distal end portion of the insertion portion 20. The treatment portion 30 of the present embodiment is an openable and closable forceps and a basic structure thereof is publically known. As a high frequency current is supplied to the treatment portion 30 with tissue being grasped by the treatment portion 30, it is possible to perform tissue ablation, coagulation, hemostasis, or the like. A power transfer member 60 for driving and opening-closing the treatment portion 30 is formed of a conductive material such as metal in the shape of a rod or a wire. The distal end portion of the power transfer member 60 is connected to the treatment portion 30. The power transfer member 60 extends to the manipulation portion 40 through the insertion portion 20.

The manipulation portion 40 includes a main body 41 to which the insertion portion is connected, a handle 42 that is to be grasped by a manipulator, and a trigger 43 and a power-on button 44 that are manipulated by the manipulator.

The power transfer member 60, which extends to an inside of the insertion portion 20 and the main body 41, is connected to the trigger 43. When the manipulator manipulates the trigger 43, the power transfer member 60 moves advanced or retracted to open or close the treatment portion 30.

The power receiver 50 includes a plate-shaped portion 51 and a planar coil 52. As shown in FIG. 2, the shape of the plate-shaped portion 51 is disk-shaped having a through-hole 51a at the center. The plate-shaped portion 51 is formed of an insulating material such as resin. The insertion portion 20 is inserted and fixed into the through-hole 51a.

The planar coil 52 is formed as a metal wire is spirally wound in a single surface. In the present embodiment, the planar coil 52 is formed as a metal wire is spirally wound around the through-hole 51a to form a surface with the through-hole 51a being centered thereon as shown in FIG. 2. The planar coil 52 is arranged in the plate-shaped portion 51 at a middle portion in the thickness direction thereof such that the planar coil 52 is not exposed from the outer surface of the plate-shaped portion 51. The plate-shaped portion 51 of the present embodiment is formed of transparent resin and therefore the planar coil 52 is visible as shown in FIGS. 1 and 2.

The power receiver 50 is electrically connected to the main body 41 via a cable 53. A first end portion of the cable 53 is electrically connected to the planar coil 52 and a second end portion thereof is electrically connected to a power connection member (not shown) in the main body 41 of the manipulator 40. The power connection member is electrically connected to the power-on button 44 and the power transfer member 60.

FIG. 3 is a side view illustrating the trocar 70. FIG. 4 is a plan view of the trocar 70. The trocar 70 includes a main body portion 71 and a power transmitter 72.

A basic structure of the main body portion 71 is the same as that of a publically known trocar. That is, the main body portion 71 is formed in a tubular shape such that a through-hole (not shown) into which the insertion portion 20 of the treatment tool 10 is insertable extends from a proximal end portion to a distal end portion of the main body portion 71. The main body portion 71 may have an inner needle or the like for opening a hole in an abdominal wall or a valve for keeping the abdomen being insufflated as needed.

The power transmitter 72 is provided at the proximal end side of the main body portion 71. The power transmitter 72 has a planar coil (i.e., a power transmitting member) 75 and a power transmission cable 76 that is electrically connected to the planar coil 75. The material and shape of the power transmitter 72 and the arrangement of the planar coil 75 are substantially the same as those of the plate-shaped portion 51 and the planar coil 52 of the power receiver 50. The planar coil 75 of the power transmitter 72 is arranged so as to be substantially parallel to a proximal end surface of the trocar 70, that is, a proximal end surface 72a of the power transmitter 72 (which can also be referred to as a power transmitting side proximal end surface), and is coated with an insulating material. The power transmitter 72 has a through-hole 72b at a central portion thereof. The through-hole 72b communicates with the through-hole of the main body portion 71 and the insertion portion 20 of the treatment tool 10 is inserted into the through-hole 72b. The power transmission cable 76 is electrically connected to a power supply source that is not shown.

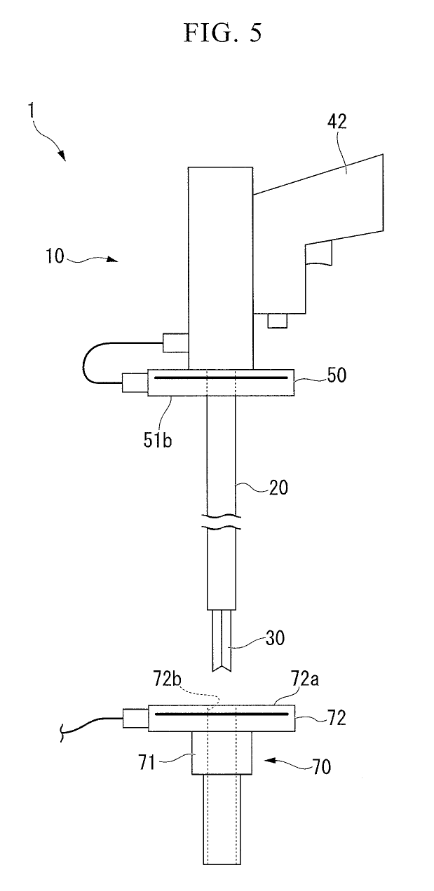

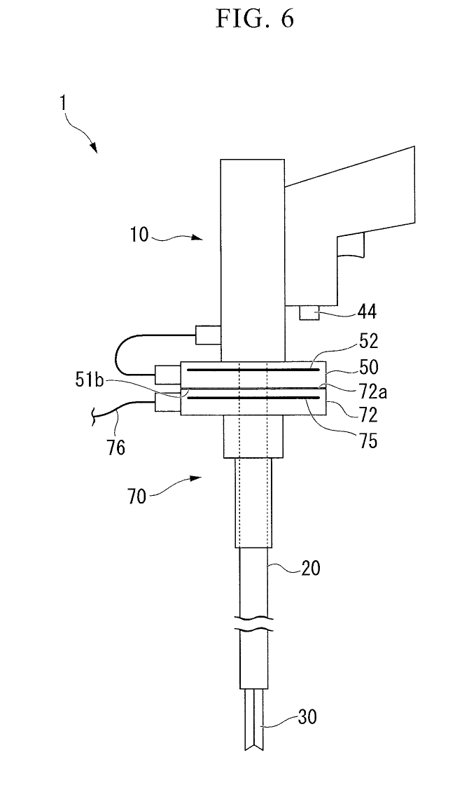

In the present embodiment, the planar coil 52 of the treatment tool 10 and the planar coil 75 of the trocar 70 are substantially the same in size as shown in FIGS. 5 and 6.

The planar coil 75 is visible since the power transmitter 72 of the present embodiment is also formed of a transparent material. However, the power receiving member and the power transmitting member may not necessarily be arranged such that they are visible and the plate-shaped portion or the power transmitter may be formed of a colored insulating material.

The movement of the medical power supply system of the present embodiment including the treatment tool 10 and the trocar 70 configured as described above operates when being in use is described below.

First, the manipulator cuts the abdominal wall of a patient or the like to form an opening into the abdominal cavity. The main body portion 71 of the trocar 70 is inserted from the distal end side thereof into the opening and the trocar 70 is placed in the abdominal wall. The power transmission cable 76 is then connected to the power supply source. The power transmission cable 76 and the power supply source may be connected either before or after the trocar 70 is placed.

FIGS. 5 and 6 are views illustrating a procedure when the medical power supply system is used. The manipulator grasps the handle 42 of the treatment tool 10 and inserts the treatment portion 30, from the proximal end side of the trocar 70, into the trocar 70 as shown in FIG. 5. The treatment portion 30 is first introduced into the main body portion 71 through the though-hole 72b of the power transmitter 72 and the insertion portion 20 is then introduced into the main body portion 71.

When the manipulator inserts the treatment tool 10 into the trocar 70 to some extent, the power transmitter 72 of the trocar 70 and the power receiver 50 are close to each other. Thus, the planar coil 75 arranged in the power transmitter 72 and the planar coil 55 arranged in the power receiver 50 face each other in the longitudinal direction of the insertion portion 20 and in radially outside the insertion portion 20. As the treatment tool 10 is further inserted (to a predetermined length), the treatment portion 30 and the insertion portion 20 are moved relative the trocar 70 toward the distal end side of the main body portion 71.

When the proximal end surface 72a (the power transmitting side proximal end surface) of the power transmitter 72 and the distal end surface 51b (the power receiving side distal end surface) of the power receiver 50 are close to each other or in contact with each other as shown in FIG. 6, the planar coil 75 of the power transmitter 72 and the planar coil 55 of the power receiver 50 are in a positional relationship such that electromagnetic coupling is possible therebetween. In this state, the manipulator can perform various treatments using the treatment tool 10.

When the manipulator desires to supply power to the treatment portion 30, the manipulator presses the power-on button 44 of the manipulation portion. Power supplied from the power source is then transmitted to the planar coil 75 via the power transmission cable 76. Power is transmitted from the power transmitter 72 to the power receiver 50, and thus received by the power receiver 50, by wireless power supply implemented as electromagnetic coupling occurs between the facing surfaces of the planar coils 75 and 52 which are arranged facing each other in the longitudinal direction of the insertion portion 20. Thereafter, power is supplied to the treatment portion 30 via the cable 43 and the power transfer member 60.

As described above, the medical power supply system according to the present embodiment is configured such that the power transmitter 72 provided in the trocar 70 and the power receiver 50 provided in the treatment tool 10 are electromagnetically coupled, outside the trocar 70 and radially outside the insertion portion 20, to perform wireless power supply. In the medical power supply system according to the present embodiment, the diameter size of an insertion portion of a medical instrument, which is inserted into the trocar 70, does not affect the transmission efficiency of wireless power supply. Therefore, even when medical instruments having insertion portions with different diameter sizes are used, it is possible to provide the same wireless power supply properties for all medical instruments. Accordingly, it is possible to uniformize the wireless power supply properties for all medical instruments while significantly improving the degree of freedom of design of medical instruments.

When at least a part of the insertion portion 20 of the treatment tool 10 is made of a magnetic member as a modified example of the above embodiment, it is possible to induce a magnetic flux between the planar coils 52 and 75 of the power receiver 50 and the power transmitter 72 along the longitudinal axis of the insertion portion 20 during wireless power supply, thereby increasing the transmission efficiency. For example, the insertion portion 20 may be made of stainless steel (SUS420). It is also possible to give magnetic properties to the insertion portion 20 by coating an outer surface of the insertion portion 20 only in a specific area thereof extending in the axial direction with a magnetic member (for example, nickel or the like). The insertion portion 20 may also be configured by arranging a soft magnetic member such as ferrite on the inner surface of a cylindrical member formed of a nonconductive material such as resin.

Instead of the insertion portion 20, even when the power transfer member 60 is configured to include a magnetic member, it is possible to induce a magnetic flux between the planar coils 52 and 75 of the power receiver 50 and the power transmitter 72 in the longitudinal direction of the insertion portion 20 to improve the transmission efficiency, similar to the modified example described above. For example, similar to the insertion portion 20 described above, the power transfer member 60 may be coated with nickel on a surface thereof or may be made of stainless steel (SUS420). A wire-shaped member utilizing a soft magnetic member such as iron, silicon steel, soft ferrite, or Permalloy may also be used as the power transfer member 60.

Both of the power transfer member 60 and the insertion portion 20 of the treatment tool 10 may be magnetic.

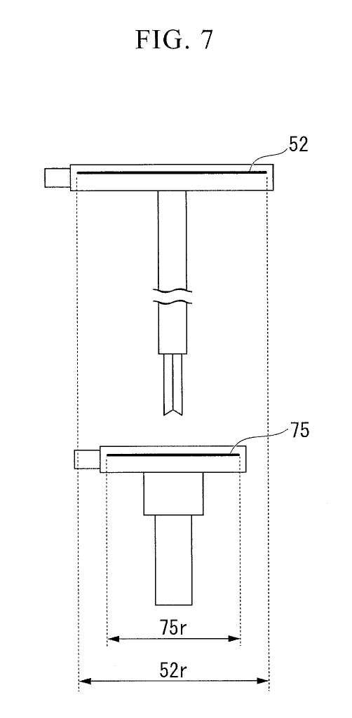

In addition, as shown in FIG. 7, a diameter size 52r of the planar coil 52 of the treatment tool 10 may be set to be greater than a diameter size 75r of the planar coil 75 of the trocar 70. That is, the area of a power receiving region (the power receiving area) of the planar coil 52 of the power receiver 50 may be set to be greater than the area of a power transmitting region (the power transmitting area) of the planar coil 75 of the power transmitter 72. This configuration can increase the transmission efficiency by increasing the magnetic flux that can be received by the power receiver 50 in the magnetic flux generated by the power transmitter 72.

In the present embodiment, the power receiver 50 and the power transmitter 72 are disc-shaped and the power receiving member and the power transmitting member are exemplified by the planar coils 52 and 75 that are formed as metal wires are spirally wound around the through-holes 51a and 72b into which the insertion portion 20 is inserted. However, the present embodiment is not limited to this example. For example, a plurality of planar coils may be provided around the though-hole 72b.

### MODIFIED EXAMPLE

Although the power receiving member and the power transmitting member are made of the planar coils 52 and 75 in the first embodiment, any power receiving and transmitting members may be employed as long as wireless power supply is possible. For example, the power receiving member and the power transmitting member may be made of planar electrodes formed of metallic foil or the like, instead of planar coils.

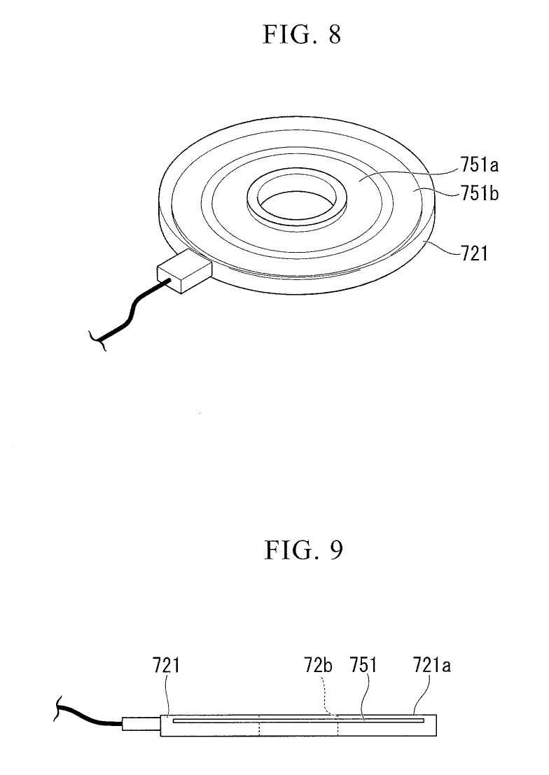

FIG. 8 is a perspective view illustrating a power transmitter 721 in the modified example. FIG. 9 is a side view of the power transmitter 721. The power transmitter 721 of this modified example has a planar electrode 751. The planar electrode 751 is at least electrically divided into two regions 751a and 751b. For example, the planar electrode 751 is divided into concentric circular rings around the through-hole 72b. The planar electrode 751 is divided such that the areas of the electrodes 751a and 751b are substantially equal. The planar electrode 751 is arranged over an area, which extends from the opening edge of the through-hole 72b, into which the insertion portion 20 of the treatment tool 10 is inserted, to near the outer periphery of the power transmitter 721, in a surface 721a of the power transmitter 721 at the proximal end side thereof and is coated with an insulating material such that the surface of the planar electrode 751 is not exposed from the outer surface. The planar electrode 751 is arranged closer to the proximal end surface of the power transmitter than when a planar coil is used. A planar electrode is also arranged in the power receiver (not shown). The material, shape, and arrangement of the planar electrode of the power receiver are substantially the same as those of the power transmitter 721.

Wireless power is supplied to planar electrodes arranged in the power receiving and transmitting portions to face each other with an insulating material provided therebetween. In this case, the planar electrodes facing each other are connected through electric field coupling. Specifically, two pairs of planar electrodes facing each other are formed between the power receiving and transmitting portions and wireless power supply from the power transmitter to the power receiver can be performed through electric field coupling between the planar electrodes facing each other. In addition, the planar electrode of each of the power transmitting and receiving portions is divided into concentric circular rings around the through-hole. Therefore, even when the power receiver moves (rotates) about the axial direction of the insertion portion relative to the power transmitter as the treatment tool is manipulated, the facing areas of the electrodes do not change, thereby enabling reliable power transmission.

In this modified example, even when medical instruments having insertion portions with different diameter sizes are used, it is possible to provide the same wireless power supply properties for all medical instruments, similar to the first embodiment. In addition, when planar electrodes are used as the power transmitting and receiving members as in this modified example, it is possible to reduce the thicknesses of the power receiving and transmitting portions.

### Second Embodiment

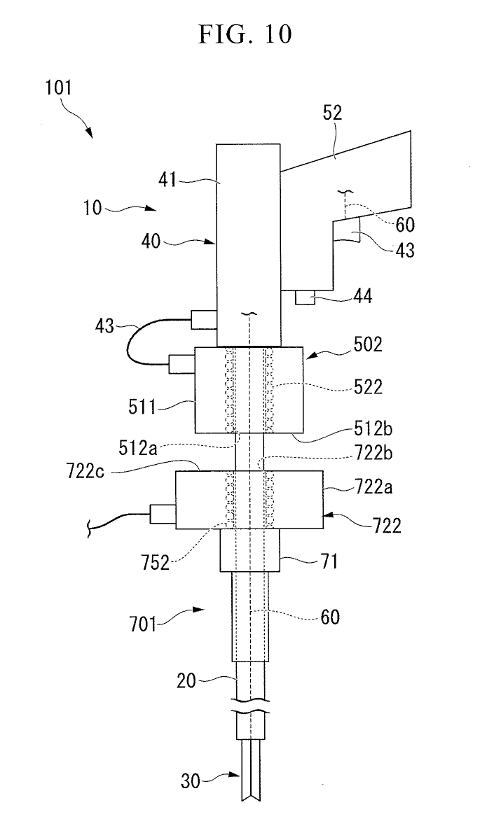

A medical power supply system 101 according to a second embodiment will now be described with reference to FIG. 10. In the embodiment described below, elements which have functions or structures similar to the elements of the medical power supply system according to the first embodiment described above are denoted by the same reference numbers as those of the first embodiment and descriptions similar to the first embodiment are omitted.

FIG. 10 is a side view illustrating a medical instrument 10 and a trocar 70 in a medical power supply system 101. The medical power supply system 101 according to the present embodiment differs from the first embodiment in the power receiver and the power transmitter.

Instead of the plate-shaped portion 51 of the first embodiment, a cylindrical portion 511 extending in the axial direction of the insertion portion 20 is provided at a distal end side of a main body 41 of a treatment tool 10. A power receiver 502 includes a power receiving coil 522 wound along the axial direction in the cylindrical portion 511 as shown in FIG. 10. The power receiving coil 522 is arranged along the axial direction of the through-hole 512a and the surface of the power receiving coil 522 is coated such that the power receiving coil 522 is not exposed from the inner circumferential surface of the through-hole 512a.

As shown in FIG. 10, a power transmitter 722 includes a cylindrical portion 722a extending in the axial direction and a power transmitting coil 752 as a power transmitting member which is wound along the axial direction around the axial line of an insertion hole 722b of a trocar 701 in the cylindrical portion 722a.

When the medical power supply system 101 according to the present embodiment is used, the treatment tool 10 is inserted into the trocar 701. As the treatment portion 30 of the treatment tool 10 protrudes from the trocar 701, a proximal end surface 722c of the power transmitter 722 (a proximal end surface at the power transmitting side) and a distal end surface 512b of the power receiver 501 (a distal end surface at the power receiving side) approach and face each other, allowing the power transmitting coil 752 and the power receiving coil 522 to be in a positional relationship such that electromagnetic coupling is possible therebetween. When the manipulator presses the power-on button 44 of the manipulation portion with the proximal end surface 722c of the power transmitter 722 and the distal end surface 512b of the power receiver 501 being close to each other or in contact with each other, wireless power supply is performed between the power transmitting coil 752 and the power receiving coil 522.

Similar to the first embodiment, the medical power supply system 101 according to the present embodiment is configured such that wireless power supply is performed by producing electromagnetic coupling, outside the trocar 701 and radially outside the insertion portion 20. Therefore, even when medical instruments having insertion portions with different diameter sizes are used, it is possible to provide the same wireless power supply properties for all medical instruments.

When at least a part of the insertion portion 20 of the treatment tool 10 is made of a magnetic member in the present embodiment, it is possible to induce a magnetic flux of the power transmitting coil 752 in the longitudinal direction of the insertion portion 20 when wireless power supply is performed, thereby increasing the transmission efficiency. When the power transfer member 60 is configured to include a magnetic member, it is also possible to increase the transmission efficiency as described above.

Although the medical instrument is exemplified by a treatment tool in each of the above embodiments, the end effector of the present invention is not limited to the forceps described above and any end effector may be used as long as it can provide a specific function when power is supplied thereto. For example, the medical instrument may be a high frequency knife that is used when current is applied thereto or an observation means that includes an image capturing element, an optical system, a lighting mechanism, or the like.

Although the guide tube of the present invention is exemplified by a trocar in each of the above embodiments, the guide tube of the present invention is not limited to the trocar described above. Thus, the present invention may be applied to an overtube which is used to introduce an endoscope or a treatment tool into a body cavity, or the like, as the guide tube.

Although the embodiments of the present invention have been described above, the technical scope of the present invention is not limited to the above embodiments, and the combinations of elements in each of the embodiments may be changed, various modifications may be made to each of the elements, and any of the elements may be removed without departing from the spirit of the present invention.

1. A medical power supply system comprising:
a medical instrument which has an elongated insertion portion that is inserted into a body, an end effector that is provided at a distal end portion of the insertion portion, and a power receiver that has a power receiving member and receives power to be supplied to the end effector; and
a guide tube which has a power transmitting member that is connected to a power source and that is provided at a proximal end side of the guide tube, and which guides the insertion portion of the medical instrument so as to be inserted from the proximal end side into the body,
wherein the power receiving member is arranged radially outside the insertion portion, and
when the insertion portion is inserted into the guide tube to a predetermined length such that the power transmitting member and the power receiving member are close to each other, a power transmitting side proximal end surface of the power transmitting member and a power receiving side distal end surface of the power receiving member face each other in an axial direction of the insertion portion and are in a positional relationship such that wireless power supply is possible.

2. The medical power supply system according to claim 1, wherein the power transmitting member and the power receiving member are each a coil and the power transmitting member and the power receiving member are electromagnetically coupled and perform wireless power supply.

3. The medical power supply system according to claim 2, wherein the power transmitting member is a planar coil provided at the power transmitting side proximal end surface, and
the power receiving member is a planar coil provided at the power receiving side distal end surface.

4. The medical power supply system according to claim 3, wherein a power receiving area of the planar coil of the power receiving member is greater than a power transmitting area of the planar coil of the power transmitting member.

5. The medical power supply system according to claim 2, wherein at least a part of the insertion portion is magnetic.

6. The medical power supply system according to claim 2, wherein the medical instrument includes a power transfer member that is inserted into the insertion portion and that activates the end effector, and
the power transfer member is configured to include a magnetic member.

7. The medical power supply system according to claim 1, wherein the power transmitting member is a planar electrode provided at the power transmitting side proximal end surface,
the power receiving member is a planar electrode provided at the power receiving side distal end surface, and
the power transmitting member and the power receiving member are electromagnetically coupled and perform wireless power supply.
